(19) United States
(12) Patent Application Publication
(10) Pub. No.: US 2026/0355414 A1
(43) Pub. Date: Nov. 12, 2026

(54) System and Method for Pressure Management for a Drug Delivery Device

(71) Applicant: Becton, Dickinson and Company, Franklin Lakes, NJ (US)

(72) Inventors: Steve Beguin, Nenzlingen (CH); Michael Yarger, Chapel Hill, NC (US); Simon O'Reilly, Dublin (IE)

(21) Appl. No.: 19/748,334

(22) Filed: Jul. 21, 2026

Related U.S. Application Data
(63) Continuation of application No. 17/527,667, filed on Nov. 16, 2021, now Pat. No. 12,708,707.
(60) Provisional application No. 63/114,905, filed on Nov. 17, 2020.

(51) Int. Cl. A61M 5/142

(52) U.S. Cl. CPC A61M 5/14244; A61M 2005/14208; A61M 2205/3331; A61M 2205/50; A61M 2205/8212

(57) ABSTRACT

A drug delivery device includes a power source, a reservoir configured to receive a fluid, a fluid line in fluid communication with the reservoir, a pump configured to deliver a fluid from the reservoir to the fluid line, and a power limitation subsystem configured to limit a power level supplied to the pump.

### CROSS-REFERENCE TO RELATED APPLICATIONS

The present application is a continuation of United States patent application Ser. No. 17/527,667, filed Nov. 16, 2021, entitled “System and Method for Pressure Management for a Drug Delivery Device”, which claims priority to U.S. Provisional Application No. 63/114,905, filed Nov. 17, 2020, entitled “System and Method for Pressure Management for a Drug Delivery Device”, the entire disclosures of each of which are hereby incorporated by reference in their entirety.

### BACKGROUND OF THE INVENTION

### Field of the Invention

The present disclosure relates to a device for pressure management for a drug delivery device.

### Description of Related Art

Wearable medical devices, such as automatic injectors, have the benefit of providing therapy to the patient at a location remote from a clinical facility and/or while being worn discretely under the patient's clothing. The wearable medical device can be applied to the patient's skin and configured to automatically deliver a dose of a pharmaceutical composition within a predetermined time period after applying the wearable medical device to the patient's skin, such as after a 27 hour delay. After the device delivers the pharmaceutical composition to the patient, the patient may subsequently remove and dispose of the device.

In certain circumstances, due to the medium in which the liquid is being injected, the flow of fluid leaving the device may be impaired, which can lead to increased pressure in the fluid line of the device. When the pressure rises above a certain threshold, the integrity of the fluid path may be compromised causing a leak within the device and a failure to deliver the full dose of medicament. A fluid leak within the device may also cause damage to the device and subsequent system failures as well as potential contamination concerns due to contact between the fluid and the device.

Human subcutaneous tissue is composed of various cell types, extracellular matrix (ECM) constituents, microstructures, and macroscopic arrangement of cells and ECM. Those elements contribute to the mechanical properties of the tissue. The tissue may also include lymphatic system and blood vessels, and has intrinsic fluid absorption and retention properties. These characteristics vary among individuals, location within the body, and over time may cause variable degrees of resistance to the infusion of fluids at the site of injection. When the resistance of the tissue is too high or the absorption rate is too low for a given delivery flow rate from the device, the pressure may build up and reach valves above the threshold where the fluid line and other components may be compromised.

### SUMMARY OF THE INVENTION

In one aspect or embodiment, a drug delivery device includes a power source, a reservoir configured to receive a fluid, a fluid line in fluid communication with the reservoir, a pump configured to deliver a fluid from the reservoir to the fluid line, and a power limitation subsystem configured to limit a power level supplied to the pump.

The power limitation subsystem may be a current limiter subsystem. The current limiter subsystem may include PNP transistors or NPN transistors. The drug delivery device may further include a microcontroller, where the power limitation subsystem includes the microcontroller configured to modulate a voltage supplied to the pump. The power limitation subsystem has an activated mode and a deactivated mode.

In a further aspect or embodiment, a method of pressure management for a drug delivery device including a microcontroller, a reservoir, a pump, a fluid line, and a power source, includes: delivering fluid through the fluid line via the pump at a first power level; detecting a pressure within the fluid line; determining whether the pressure within the fluid line exceeds a high pressure threshold level; delivering fluid through the fluid line via the pump at a second power level until a predetermined condition is satisfied, where the second power level is lower than the first power level; and resuming the delivery of the fluid through the fluid line at the first power level after the predetermined condition is satisfied.

The predetermined condition may be a predetermined pressure level within the fluid line. The second power level may be provided via a current limiter subsystem. The current limiter subsystem may include transistors, such as bipolar, MOSFET, or CMOS transistors, an operational amplifier, or other active circuitry. The second power level may be provided by modulating the voltage supplied to the pump. The pressure within the fluid line may be detected by measuring a current of the drug delivery device during actuation of the pump. The measuring of the current of the drug delivery device may include subtracting a reference current value from a peak current value during an actuation cycle of the pump to determine a stroke current value. The power may be controlled by modulating or controlling a level of current or by modulating or controlling a level of voltage.

In a further aspect or embodiment, a computer program product for a method of pressure management for a drug delivery device including a microcontroller, a reservoir, a pump, a fluid line, and a power source, the computer program product including at least one non-transitory computer-readable medium including program instructions that, when executed by the microcontroller, cause the drug delivery device to: deliver fluid through the fluid line via the pump at a first power level; detect a pressure within the fluid line; determine whether the pressure within the fluid line exceeds a high pressure threshold level; deliver fluid through the fluid line via the pump at a second power level until a predetermined condition is satisfied, with the second power level lower than the first power level; and resume the delivery of the fluid through the fluid line at the first power level after the predetermined condition is satisfied.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a drug delivery device according to one aspect or embodiment of the present application.

FIG. 2 is a perspective view of the drug delivery device of FIG. 1, with a top cover removed.

FIG. 3 is a schematic of the drug delivery device of FIG. 1.

FIG. 4 is a graph of current versus time for the drug delivery device of FIG. 1, showing a 0 psi pressure condition according to one aspect or embodiment of the present application.

FIG. 5 is a graph of current versus time for the drug delivery device of FIG. 1, showing a 40 psi pressure condition according to one aspect or embodiment of the present application.

FIG. 6 is a schematic of a current limiting circuit according to one aspect or embodiment of the present application.

FIG. 7 is a schematic of a current limiting circuit according to a second aspect or embodiment of the present application.

FIG. 8 is a schematic of a current limiting circuit according to a third aspect or embodiment of the present application.

FIG. 9 is a schematic of a method of modulating power according to a further aspect or embodiment of the present application.

FIG. 10A is a graph of current versus time for the drug delivery device of FIG. 1, showing a method of determining fluid path pressure according to one aspect or embodiment of the present application.

FIG. 10B is an enlarged graph of area 10B shown in FIG. 10A.

FIG. 10C is an enlarged graph of area 10C shown in FIG. 10B.

FIG. 11 is a schematic of a method of power management for drug delivery device according to one aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

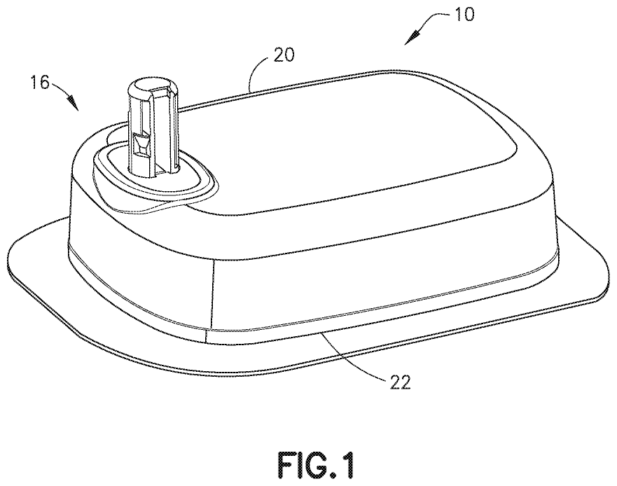

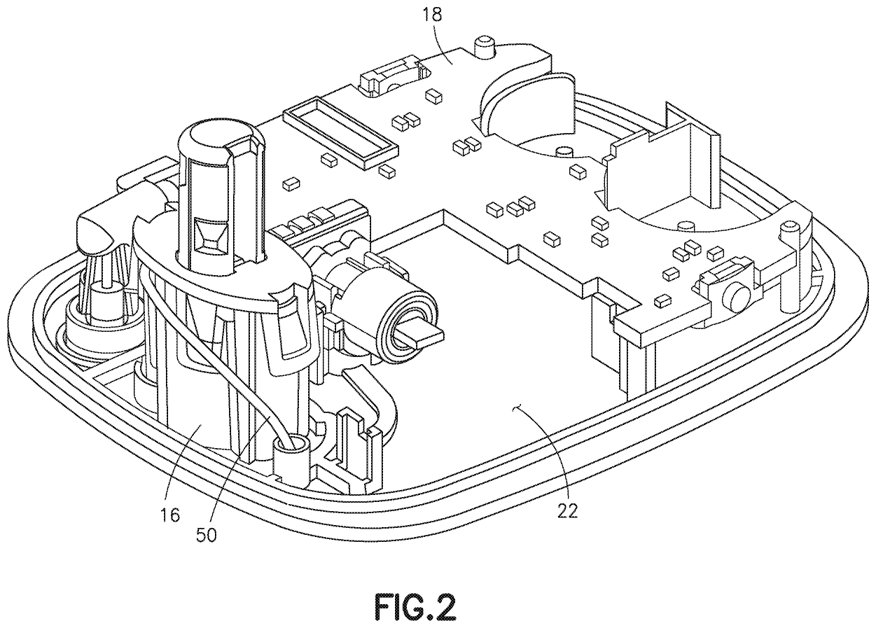

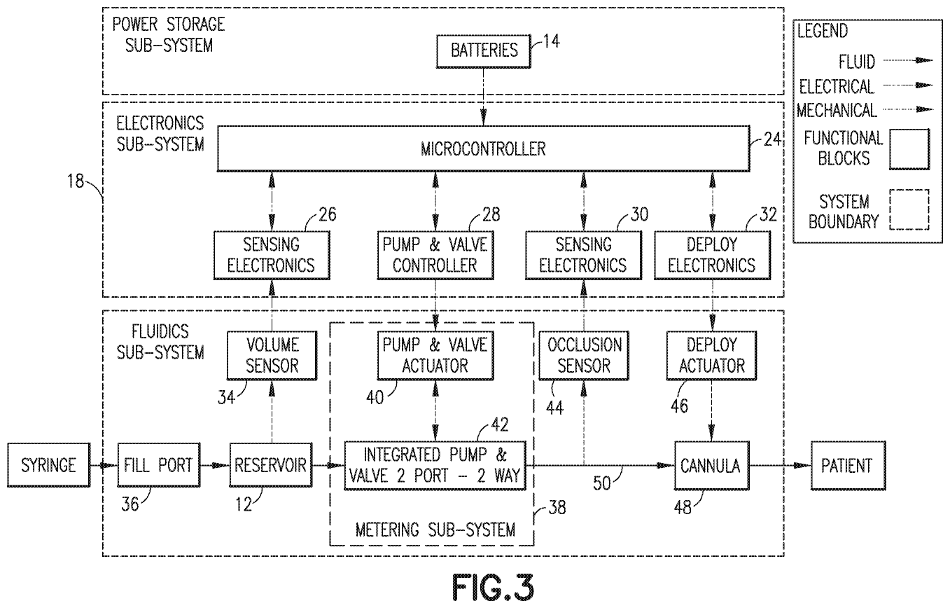

Referring to FIGS. 1-3, a drug delivery device 10 includes a reservoir 12, a power source 14, an insertion mechanism 16, control electronics 18, a cover 20, and a base 22. In one aspect or embodiment, the drug delivery device 10 is a wearable automatic injector, such as an insulin or bone marrow stimulant delivery device. The drug delivery device 10 may be mounted onto the skin of a patient and triggered to inject a pharmaceutical composition from the reservoir 12 into the patient. The drug delivery device 10 may be pre-filled with the pharmaceutical composition, or it may be filled with the pharmaceutical composition by the patient or medical professional prior to use.

The drug delivery device 10 is configured to deliver a dose of a pharmaceutical composition, e.g., any desired medicament, into the patient's body by a subcutaneous injection at a slow, controlled injection rate. Exemplary time durations for the delivery achieved by the drug delivery device 10 may range from about 5 minutes to about 60 minutes, but are not limited to this exemplary range. Exemplary volumes of the pharmaceutical composition delivered by the drug delivery device 10 may range from about 0.1 milliliters to about 10 milliliters, but are not limited to this exemplary range. The volume of the pharmaceutical composition delivered to the patient may be adjusted.

Referring again to FIGS. 1-3, in one aspect or embodiment, the power source 14 is a DC power source including one or more batteries. The control electronics 18 include a microcontroller 24, sensing electronics 26, a pump and valve controller 28, sensing electronics 30, and deployments electronics 32, which control the actuation of the drug delivery device 10. The drug delivery device 10 includes a fluidics sub-system that includes the reservoir 12, a volume sensor 34 for the reservoir 12, a reservoir fill port 36, and a metering system 38 including a pump and valve actuator 40 and a pump and valve mechanism 42. The fluidic sub-system may further include an occlusion sensor 44, a deploy actuator 46, a cannula 48 for insertion into a patient's skin, and a fluid line 50 in fluid communication with the reservoir 12 and the cannula 48. In one aspect or embodiment, the insertion mechanism 16 is configured to move the cannula 48 from a retracted position positioned entirely within the device 10 to an extended position where the cannula 48 extends outside of the device 10. The drug delivery device 10 may operate in the same manner as discussed in U.S. Pat. No. 10,449,292 to Pizzochero et al, incorporated herein by reference.

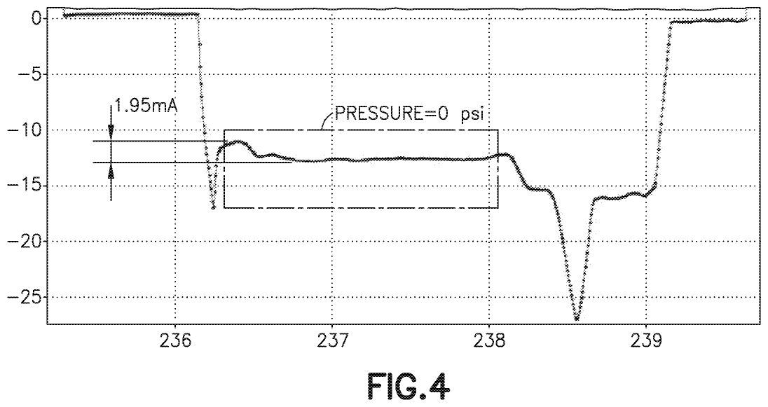

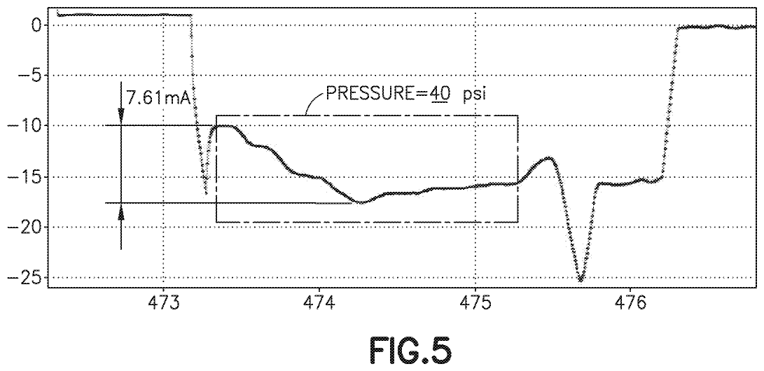

Referring to FIGS. 4 and 5, a relationship between pressure within the fluid line 50 and current required to push the pump and valve mechanism 42 forward is shown. The pump and valve mechanism 42 has an aspiration cycle and a dispense cycle with the dispense cycle being shown in FIGS. 4 and 5. As shown in FIG. 4, with a current of 1.95 mA, the pressure within the fluid line 50 can be estimated to be approximately 0 psi. As shown in FIG. 5, with a current of 7.61 mA, the pressure within the fluid line 50 can be estimated to be approximately 40 psi. The correlation between the current and the pressure within the fluid line 50 may be determined via testing using a pressure sensor to measure the pressure within the fluid line 50.

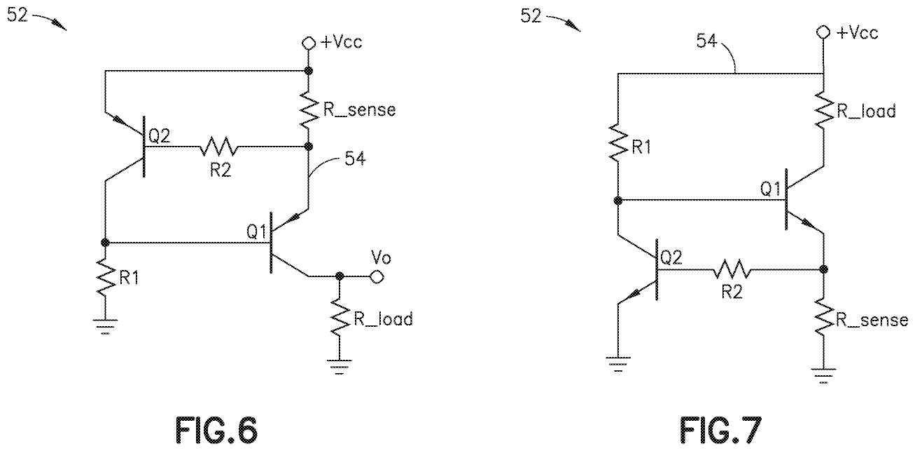

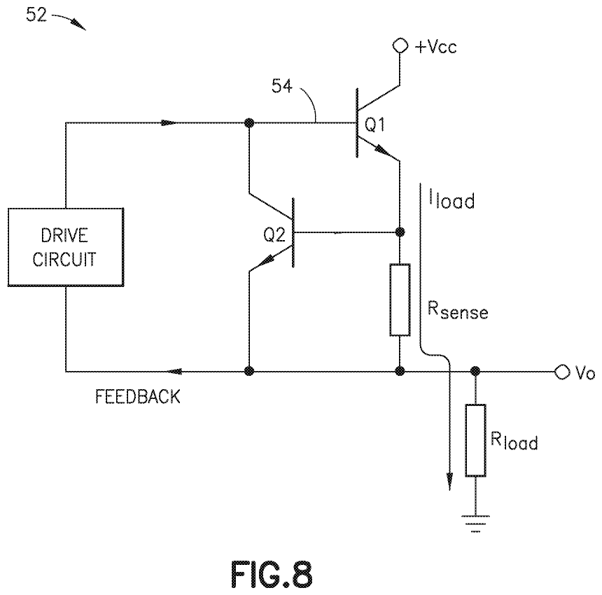

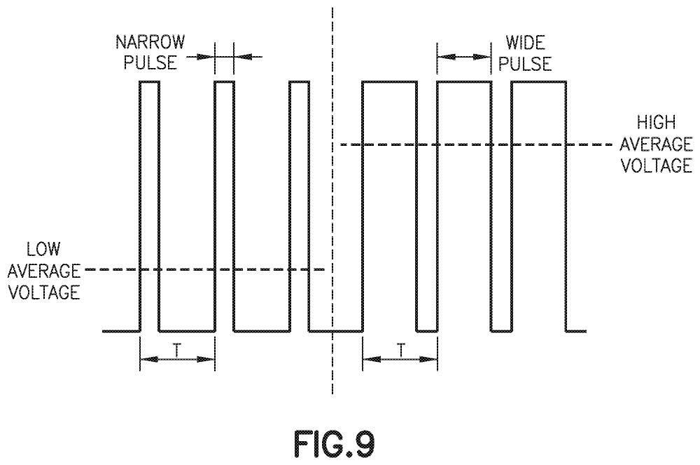

Referring to FIGS. 6-9, in one aspect or embodiment, the drug delivery device 10 includes a power limitation subsystem 52 configured to limit a power level supplied to the pump and valve mechanism 42. As shown in FIGS. 6-8, the power limitation subsystem 52 may be a current limiter subsystem 54. The current limiter subsystem 54 is configured to limit or cap the current supplied to the pump and valve mechanism 42. The current limiter subsystem 54 may utilize PNP transistors (FIG. 6) and/or NPN transistors (FIGS. 7 and 8). In a further aspect or embodiment, the power limitation subsystem 52 is provided by the microcontroller 24 modulating a voltage supplied to the pump and valve mechanism 42. As shown in FIG. 9, by using pulse width modulation, the power supplied to the pump and valve mechanism 42 can be modulated. For example, using a narrow pulse over a period of time will result in a lower average voltage than using a wider pulse over a period of time. The pulse width modulated signal may be smoothed by using dedicated circuitry such as capacitors, or may be smoothed by the load formed by the actuator itself.

In one aspect or embodiment, the power limitation subsystem 52 is configured to be adjustable to allow the power level supplied to the pump and valve mechanism 42 to be varied as needed. In one aspect or embodiment, the power limitation subsystem 52 has an activated mode where the power level being supplied is limited and a deactivated mode where the power level being supplied is not limited. The activated mode and the deactivated mode may be provided via additional circuitry and/or by the control via the microcontroller 24.

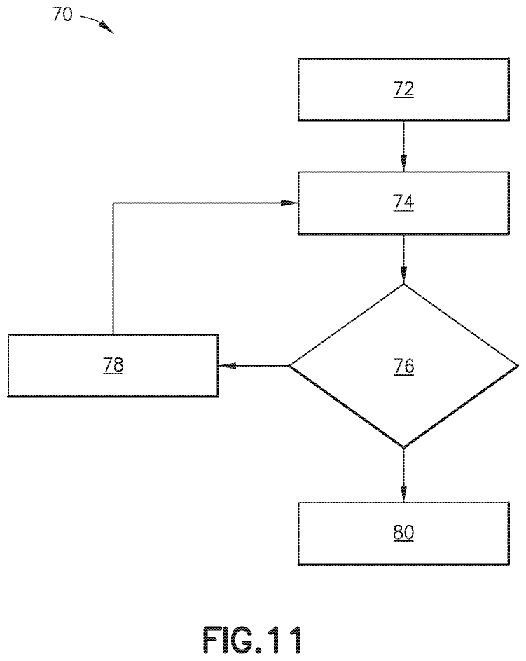

Referring to FIG. 11, in one aspect or embodiment, a method 70 of pressure management for the drug delivery device 10 includes: delivering fluid 72 through the fluid line 50 via the pump and valve mechanism 42 at a first power level; detecting 74 a pressure within the fluid line 50; determining 76 whether the pressure within the fluid line 50 exceeds a high pressure threshold level; delivering fluid 78 through the fluid line 50 via the pump and valve mechanism 42 at a second power level until a predetermined condition is satisfied, where the second power level is lower than the first power level; and resuming 80 the delivery of the fluid through the fluid line 50 at the first power level after the predetermined condition is satisfied. In one aspect or embodiment, the predetermined condition is a predetermined pressure level within the fluid line. The second power level may be provided via the current limiter subsystem 54, as discussed above. The second power level may also be provided by modulating the voltage supplied to the pump, as discussed above.

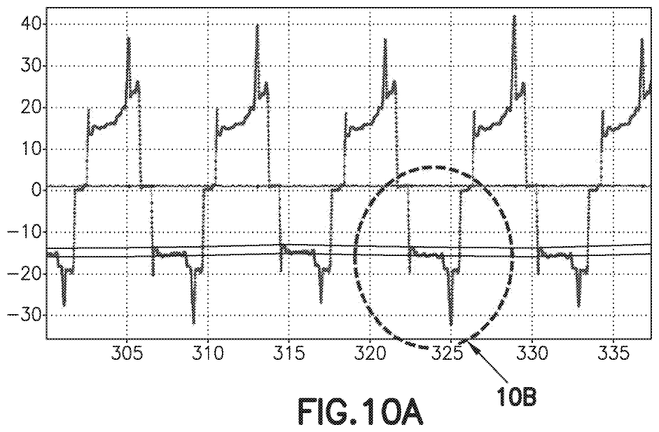

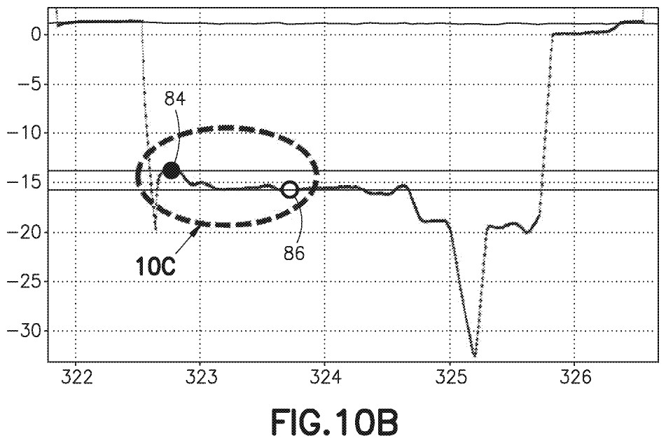

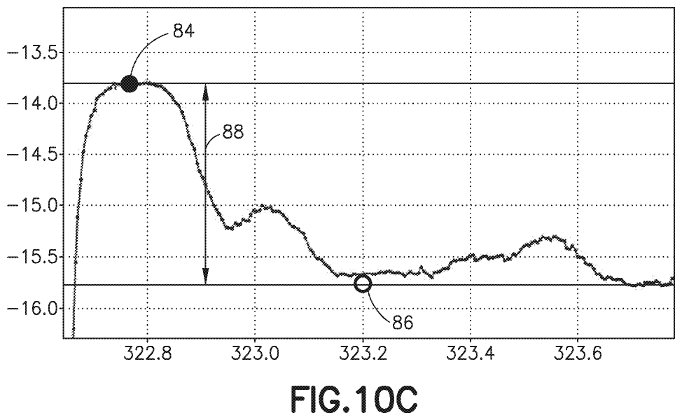

Referring to FIGS. 10A-10C, in one aspect or embodiment, the pressure within the fluid line 50 is detected by measuring a current of the drug delivery device 10 during actuation of the pump and valve mechanism 42. In one aspect or embodiment, the current is measured by measuring a voltage drop across a resistor. The measuring of the current of the drug delivery device 10 includes subtracting a reference or baseline current value 84 from a peak current value 86 during an actuation cycle of the pump and valve mechanism 42 to determine a stroke current value 88, although other suitable current detection arrangements may be utilized. The stroke current value 88 is utilized to estimate the downstream pressure of the fluid line 50 for the particular actuation cycle of the pump and valve mechanism 42. For example, the stroke current value 88 can be corresponded to various downstream pressure levels through testing or benchmarking such that the stroke current value 88 can be used to accurately estimate the pressure level of the fluid line 50.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is

1. A drug delivery device comprising:
a power source;
a reservoir configured to receive a fluid;
a fluid line in fluid communication with the reservoir;
a pump configured to deliver a fluid from the reservoir to the fluid line; and
a power limitation subsystem configured to limit power made available to the pump.

2. The drug delivery device of claim 1, wherein the power limitation subsystem comprises a current limiter subsystem.

3. The drug delivery device of claim 2, wherein the current limiter subsystem comprises transistors or an operational amplifier.

4. The drug delivery device of claim 1, further comprising a microcontroller, wherein the power limitation subsystem comprises the microcontroller configured to modulate the power made available to the pump.

5. The drug delivery device of claim 4, wherein the power is controlled by modulating or controlling a level of current.

6. The drug delivery device of claim 4, wherein the power is controlled by modulating or controlling a level of voltage.

7. The drug delivery device of claim 1, wherein the power limitation subsystem has an activated mode and a deactivated mode.

8. The drug delivery device of claim 1, wherein the power limitation subsystem is configured to limit power made available to the pump having a first power level and a second power level, the second power level being higher than the first power level, wherein the pump delivers fluid through the fluid line via the pump at the second power level until a high-pressure threshold level is reached and then delivery of the fluid through the fluid line at the first power level.

9. The drug delivery device of claim 1, wherein the power limitation subsystem is configured to limit power made available to the pump based on a pressure within the fluid line detected by determining a stroke current value subtracting a reference current value from a peak current value during an actuation cycle of the pump.
